(19) United States
(12) Patent Application Publication
(10) Pub. No.: US 2003/0212144 A1
(43) Pub. Date: Nov. 13, 2003

(54) Method for inducing an estrous cycle and pregnancy in a female mammal

(76) Inventors: Sheba J.J. MohanKumar, Okemos, MI (US); Puliyur S MohanKumar, Okemos, MI (US)

Correspondence Address: MCLEOD & MOYNE, P.C., 2190 COMMONS PARKWAY, OKEMOS, MI, 48864, US

(21) Appl. No.: 10/332,647

(22) PCT Filed: Jul. 18, 2001
(86) PCT No.: PCT/US01/22627

(51) Int. Cl. A61K 31/137

(52) U.S. Cl. 514/649

(57) ABSTRACT

The present invention provides a method for inducing an estrous cycle in a female mammal, including humans and non-human (lower) mammals with a monoamine oxidase inhibitor such as deprenyl wherein the estrous cycle of the female mammal had been inhibited by the regular treatment for a period of time whih an estrous cycle-inhibiting composition, which has been discontinued leaving the female mammal unable to ovulate, prior to impregnating with sperm. In particular, the present invention provides a method for inducing heat in a canine, particularly a retired racing greyhound to induce pregnancy. Thus, the object of the present invention is to provide a method that restores reproduction in domestic animals, in comparison animals, in captive wild animals, and in human females.

### CROSS-REFERENCE TO RELATED APPLICATION

Priority is claimed to Provisional Application No. 60/219,236, filed Jul. 18, 2000.

### STATEMENT REGARDING FEDERALLY SPONSORED RESEARCH OR DEVELOPMENT

Not Applicable.

### REFERENCE TO A “COMPUTER LISTING APPENDIX SUBMITTED ON A COMPACT DISC”

Not Applicable.

### BACKGROUND OF THE INVENTION

(1) Field of the Invention

The present invention relates to a method for inducing an estrous cycle in a female mammal, including humans and non-human (lower) mammals with a monoamine oxidase inhibitor such as deprenyl wherein the estrous cycle of the female mammal had been inhibited by the regular treatment for a period of time with an estrous cycle-inhibiting composition, which has been discontinued leaving the female mammal unable to ovulate, prior to impregnating with sperm. In particular, the present invention relates to a method for inducing heat in a canine, particularly a retired racing greyhound to induce pregnancy. Thus, the object of the present invention relates to a method that uses deprenyl or its analogs isomers, or salts to restore reproduction in domestic animals, in companion animals, in captive wild animals, and in human females wherein reproduction had been suspended for a period of time by treatment with a reproduction-inhibiting composition, which has been discontinued prior to treatment.

(2) Description of Related Art

Deprenyl is presently marketed as Anipryl™ by Pfizer Animal Health for correcting cognitive dysfunction in older animals.

The related art in the general use of deprenyl is as follows:

U.S. Pat. No. 6,033,682 to DiSanto, U.S. Pat. No. 5,981,598 to Tatton, U.S. Pat. No. 5,844,003 to Tatton, U.S. Pat. No. 5,565,495 to Stevens et al., U.S. Pat. No. 5,151,449 to Milgram et al., U.S. Pat. No. 5,276,057 to Milgram et al., Kitani et al., Life Sci. 52: 281-288 (1993); Knoll et al., Life Sci. 54: 1047-1057 (1994); MohanKumar et al., Life Sci. 61: 1783-1788 (1997); MohanKumar et al., Life Sci. 54: 841-845 (1994); Thyagarajan et al., Endocrinol. 136: 1103-1110 (1995).

There is a need for a safe and effective method for stimulating or enhancing the reproductive ability of female mammals that have been subjected to prolonged exposure to a reproduction-suppressing treatment. For example, human females who have been on oral contraceptives for a long period of time can experience inhibited or impaired ovulation after discontinuing the oral contraceptive. As a result human females may undergo the stress and expense of fertility treatments which are not always successful or can result in abnormal pregnancies. Similar problems can occur in female mammals that have been on a regimen that inhibits estrous.

For example, in racing Greyhound bitches, estrus (heat) is suppressed by treatment with testosterone. When these animals, especially the ones with superior racing performance, retire from racing, it becomes desirable to place them in a breeding program to make use of their genetic potential. However, due to prolonged exposure to testosterone, these bitches are slow to return to estrus (heat) and exhibit other reproductive problems.

Over the years, a variety of hormonal treatments have been tried to return the greyhound bitches to estrus (Bell et al. In: Proceedings of the VII World Congress Fert. Steril. 598-599 (1971); Wright. In: Proceedings of the 7^{th} Internat. Cong. Anim. Reprod. Artif. Insem. 3: 1075-1079 (1972); Thun et al., Am. J. Vet. Res. 38: 483-486 (1977); Paisley et al., J. Am. Vet. Med. Assoc. 171: 181-185 (1977); Wright, Aust. Vet. J. 56: 137-140 (1980); Allen, J. Small Anim. Pract. 28: 223-231 (1982); Chakraborty et al., Vet. Clin. North. Am. [Small Anim. Pract.] 12: 85-92 (1982); Shille et al., J. Am. Vet. Med. Assoc. 184: 1469-1473 (1984); Moses et al., J. Am. Vet. Med. Assoc. 192: 1541-1545 (1988); Cain et al., Am. J. Vet. Res. 49: 1993-1996 (1988)). However, none of the above methods have produced a reliable and practical method for inducing heat in retired Greyhound bitches.

Therefore, there remains a need for a practical and effective method for reinstating fertility or estrous in a human and mammalian female in which treatment with a fertility-inhibiting composition for an extended period of time has been discontinued.

### SUMMARY OF THE INVENTION

The present invention provides a method for inducing an estrous cycle in a female mammal, including humans and non-human (lower) mammals with a monoamine oxidase inhibitor such as deprenyl wherein the estrous cycle of the female mammal had been inhibited by the regular treatment for a period of time with an estrous cycle-inhibiting composition, which has been discontinued leaving the female mammal unable to ovulate, prior to impregnating with sperm. In particular, the present invention provides a method for inducing heat in a canine, particularly a retired racing greyhound to induce pregnancy. Thus, the object of the present invention is to provide a method that uses deprenyl or its analogs, isomers, and salts to restore reproduction in domestic animals, in companion animals, in captive wild animals, and to human females wherein reproduction had been suspended for a period of time by treatment with a reproduction-inhibiting composition, which has been discontinued prior to treatment.

Therefore, the present invention provides a method for inducing pregnancy in a female mammal wherein the estrous cycle of the female mammal has been inhibited by regular treatment for a period of time with an estrous cycle-inhibiting composition to inhibit the pregnancy, which has been discontinued leaving the female mammal unable to become pregnant, the steps comprising (a) orally administering an effective amount of a composition comprising deprenyl, an optimal isomer thereof, or a salt thereof to the female mammal to reinitiate the estrous cycle in the female mammal; and (b) impregnating the female mammal with sperm to induce the pregnancy.

In one embodiment of the method, the mammal is a human and in another embodiment, the mammal is a non-human (lower) mammal.

The present invention further provides a method for bringing a bitch canine into heat for breeding wherein the estrous cycle of the canine bitch had been inhibited by the regular treatment for a period of time with an estrous cycle-inhibiting composition which has been discontinued leaving the canine bitch unable to breed, which comprises (a) orally administering a heat-inducing amount of a composition comprising deprenyl, an optimal isomer thereof, or a salt thereof to the canine bitch to bring the bitch canine into heat; and (b) breeding the canine bitch.

In one embodiment of the method, the bitch canine is a female racing greyhound, preferably, wherein the racing greyhound has been retired from racing. In a preferred embodiment, the deprenyl is administered at a dosage of at least about 0.1 mg/kg body weight or the deprenyl is administered at a dosage not more than about 1 mg/kg of body weight.

The present invention further provides that in a method for regulating the estrus cycle in a female mammal which had been regularly administered an estrus cycle-inhibiting composition to inhibit the estrus cycle in the female mammal for a period of time and which has been discontinued leaving the female mammal unable to become pregnant, the improvement comprises orally administering an effective amount of a composition comprising deprenyl, an optimal isomer thereof, or a salt thereof to the female mammal to reinitiate the estrous cycle in the female mammal.

In one embodiment of the method the mammal is a human and in another embodiment, the mammal is a mammal other than a human. In a further embodiment, the mammal is a canine and in an embodiment further still, the canine is a racing greyhound or a racing greyhound which has been retired from racing.

In a preferred embodiment, the deprenyl is administered at a dosage of at least about 0.1 mg/kg body weight or the deprenyl is administered at a dosage not more than about 1 mg/kg of body weight.

The present invention further still provides that in a method for reinitiating a reproductive cycle in a female mammal wherein the reproductive cycle has been inhibited by treatment with an estrous cycle-inhibiting composition for a period of time and which has been discontinued leaving the female mammal unable to become pregnant, the improvement comprises orally administering an effective amount of a composition comprising deprenyl, an optimal isomer thereof, or a salt thereof to the female mammal to reinitiate the reproductive cycle in the female mammal.

In one embodiment of the method the mammal is a human and in another embodiment, the mammal is a mammal other than a human. In a further embodiment, the mammal is a canine and in an embodiment further still, the canine is a racing greyhound or a racing greyhound which has been retired from racing.

In a preferred embodiment, the deprenyl is administered at a dosage of at least about 0.1 mg/kg body weight or the deprenyl is administered at a dosage not more than about 1 mg/kg of body weight.

The present invention further still provides that in a method for reinitiating a reproductive cycle in a female mammal wherein the reproductive cycle has been inhibited by treatment with an estrous cycle-inhibiting composition for a period of time and which has been discontinued leaving the female mammal unable to become pregnant, the improvement comprises orally administering an effective amount of a composition comprising one ore more monoamine oxidase (MAO) inhibitors to the female mammal to reinitiate the reproductive cycle in the female mammal.

In one embodiment of the method the mammal is a human and in another embodiment, the mammal is a mammal other than a human. In a further embodiment, the mammal is a canine and in an embodiment further still, the canine is a racing greyhound or a racing greyhound which has been retired from racing.

In a further embodiment, the monoamine oxidase (MAO) inhibitor is selected from deprenyl, selegiline, selegiline hydrochloride, and phenethylene, N, alpha-dimethyl-N-2-propynyl hydrochloride, and iproniazid. In particular, when the monoamine oxidase (MAO) inhibitor is deprenyl, the deprenyl is administered at a dosage of at least about 0.1 mg/kg body weight or the deprenyl is administered at a dosage not more than about 1 mg/kg of body weight.

### OBJECTS

Therefore, the object of the present invention is to provide a practical and effective method that reinstates the estrous cycle in a female mammal in which treatment of the female mammal with a composition that had impaired estrous cyclicity has been discontinued.

### BRIEF DESCRIPTION OF DRAWINGS

FIG. 1 is a schematic flow diagram showing the action of deprenyl.

FIG. 2 is a graph showing bitch greyhounds in heat as a function of deprenyl, L(−) enantiomer.

FIG. 3A is a bar graph showing the effects of deprenyl (DEP) on the average release of norepinephrine (NE) from the hypothalamus in vitro. The hypothalami were incubated in Krebs-Ringers Henseleit (KRH) for four consecutive 60 min periods: with KRH alone in the first period, with KRH and deprenyl in the second period and again with KRH in the third period, and with high K+ KRH in the fourth period. At the end of each period the incubation medium was removed for neurotransmitter analysis by HPLC-EC and the hypothalami were rinsed with KRH. At the end of the fourth incubation period the hypothalami were removed and stored in 0.1 M HClO₄ and analyzed for neurotransmitter concentrations. * significantly different from NE levels in the corresponding group (10 mM) in the first incubation period (KRH) (p<0.05); ** significantly different from NE levels in the corresponding group in the first incubation period and also from other groups during the second incubation period (KRH+DEP) (p<0.01).

FIG. 3B is a bar graph showing the average content of NE in the hypothalami at the end of the experiment of FIG. 3A. * significantly different from 0 and 0.1 mM (p<0.05).

FIG. 4A is a bar graph that shows the effects of deprenyl (DEP) on the average release of dopamine (DA) from the hypothalamus in vitro. * significantly different from NE levels in the corresponding groups (1 and 10 mM) in the first incubation period (KRH) (p<0.05). See FIG. 3A legend for details.

FIG. 4B is a bar graph that shows the average content of DA in the hypothalami at the end of the experiment of FIG. 4A. * significantly different from 0 mM (p<0.05). See FIG. 3A legend for details.

FIG. 5A is a bar graph that shows the effects of 0, 0.1, 1, and 10 mM deprenyl (DEP) on the average release of dihydroxyphenylacetic acid (DOPAC) from the hypothalamus in vitro. * significantly different from DOPAC levels in the corresponding group during the first incubation period (KRH); ** significantly different from DOPAC levels in the corresponding group (10 mM) in the first incubation period (KRH) and also from other groups within the same incubation period (p<0.01). See FIG. 3A legend for details.

FIG. 5B is a bar graph that shows the average content of DOPAC in the hypothalami at the end of the experiment of FIG. 5A. * significantly different from 0 and 0.1 mM (p<0.05). See FIG. 3A legend for details.

FIG. 6A is a bar graph that shows the effects of deprenyl (DEP) on the average release of serotonin (5-HT) from the hypothalamus in vitro. * significantly different from corresponding group in the first incubation period (KRH) and also from other groups within the same incubation period (KRH+DEP) (p<0.05); ** significantly different from corresponding groups in the first incubation period (KRH)and also from 0 and 0.1 mM groups within the same incubation period (KRH+DEP) (p<0.05). See FIG. 3A legend for details.

FIG. 6B shows a bar graph of the average content of 5-HT in the hypothalami at the end of the experiment of FIG. 6A. * significantly different from 0 and 0.1 mM (p<0:05). See FIG. 3A legend for details.

FIG. 7A is a bar graph that shows the effects of deprenyl (DEP) on the average release of 5-hydroxyindoleacetic acid (5-HIM) from the hypothalamus in vitro. * significantly different from corresponding groups in other incubation periods and also from other groups within the same incubation period (p<0.01); ** significantly different from 5-HIAA levels in the first incubation period (KRH) (p<0.05). See FIG. 3A legend for details.

FIG. 7B is a bar graph that shows the average content of 5-HIAA in the hypothalami at the end of the experiment of FIG. 7A. * significantly different from 0 and 0.1 mM (p<0.05). See FIG. 3A legend for details.

### DESCRIPTION OF PREFERRED EMBODIMENTS

All patents, patent applications, government publications, government regulations, and literature references cited in this specification are hereby incorporated herein by reference in their entirety. In case of conflict, the present description, including definitions, will control.

Reproduction in the bitch greyhound and other female mammals, including female humans, is regulated by a complex neuroendocrine mechanism involving the brain, pituitary gland, and ovary. The brain, in particular the hypothalamus, through various neurotransmitters, in particular the catecholamines, regulates the secretion of a hypothalamic hormone called gonadotropin-releasing hormone (GnRH). The GnRH regulates secretion of the follicle-stimulating hormone (FSH) and the luteinizing hormone (LH) from the pituitary gland. FSH and LH act on the ovary to cause production of ova and secretion of the female sex hormones, estrogen and progesterone. The female sex hormones, especially estrogen, act on the brain to produce sexual behavior including the period of estrus (heat) during which the animals mate.

Any disturbance in the delicately balanced chain of events (catecholamines---> GnRH----> FSH, LH----> Estrogen, Progesterone) leads to disturbances in the reproductive cycle which leads to suppression of heat and ovulation. When female dogs are treated regularly with testosterone (or estrogen or progesterone), the treatment produces disturbances in neurotransmitter metabolism in the brain, which includes causing a deficiency in catecholamines in the hypothalamus. This starts a cascade of alterations in the secretion of GnRH, FSH, and LH which ultimately leads to suppression of heat, disruption of the estrous cycle, and prevention of ovulation. The birth control pill, which contains various combinations of estrogen, progesterone, and testosterone, inhibits fertility in a similar manner.

Catecholamine deficiencies can also occur under natural conditions. For example, it has been documented that when the laboratory animals such as rats or mice get old, they develop a catecholamine deficiency in the hypothalamus, which eventually leads to suppression of heat and ovulation and the disappearance of the reproductive cycle. If catecholamine deficiency in these acyclic old rats is removed by treatment with the catecholamine precursor, 1-dopa, the treatment corrects the imbalance in the secretion of various hormones and leads to restoration of heat and the estrous cycle (Quadri et al., Neuroendocrinol. 11: 248-255 (1973) Linnoile et al., J. Pharm. Esp. Ther. 199: 477-482 (1976); Huang et al., Neuroendocrinol. 20: 31-34 (1976); Cooper et al., Neuroendocrinol. 28: 234-240 (1979); and Watkins et al., Neuroendocrinol. 19: 331-338 (1975)).

Deprenyl has also been shown to restore heat and the estrous cycle in old acyclic rats (Thyagarajan et al., Endocrinol. 136: 1103-1110 (1995)). Deprenyl is the common name for phenethylamine, N, alpha-dimethyl-N-2-propynyl (or benzeneethanamine, N, alpha-dimethyl-N-2-propynyl), CAS Registry No. 2323-36-6, which is a compound that inhibits the activity of an enzyme called monoamine oxidase (MAO). MAO's function is to cause degradation of catecholamines. Deprenyl treatment inhibits MAO, and thereby increases catecholamines in the hypothalamus and other parts of the brain. This restores the normal secretion of various reproductive hormones which in turn leads to restoration of heat and estrous cycles. In another study, deprenyl has been found to increase sexual activity in old male rats (Knoll et al., Life Sci. 45: 525-532 (1989)).

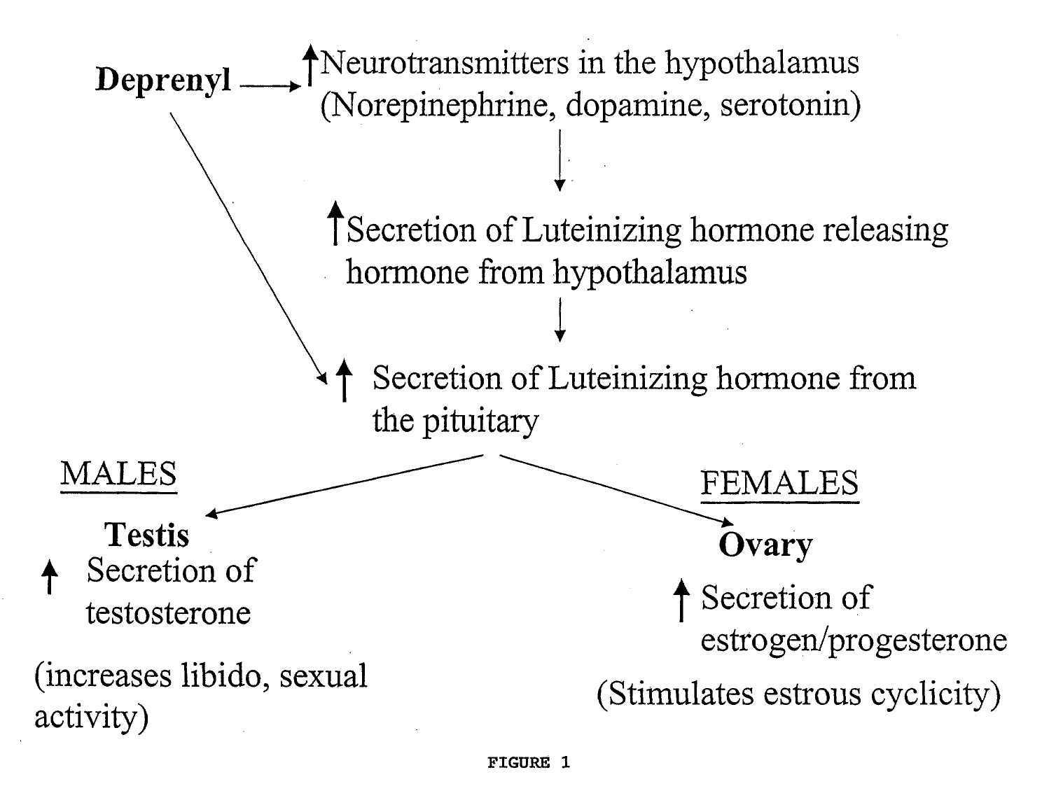

FIG. 1 is a schematic that shows the effect of deprenyl on reproduction. Deprenyl causes a dose-dependent increase in the release of neurotransmitters in the brain, e.g., norepinephrine, dopamine, and serotonin, which causes a increase in the secretion of LH from the hypothalamus. The LH from the hypothalamus along with deprenyl further increases the secretion of LH from the pituitary gland. In females, the LH causes an increase in the secretion of estrogen/progesterone in the ovary, which stimulates estrous cyclicity. In males, the LH causes a increase in the secretion of testosterone from the testis, which causes an increase in libido and sexual activity.

Over the years, a number of methods have been tried to induce heat in female dogs such as racing greyhounds, which have been treated with testosterone (or estrogen or progesterone) over a period of time to inhibit estrous. All of these methods have been focused on the use of various hormones, such as GnRH, FSH, LH, PMSG (pregnant mare serum gonadotropin) and HCG (human chorionic gonadotropin), to reinitiate the estrous cycle. While various degrees of success have been reported using the hormone-based methods, the hormone-based methods have not proved to be practical or reliable. For example, Bruyette et al. was unable to induce heat in greyhound bitches with the use of GnRH (personal communication). All of the hormones which have been tried either act on either the ovary (FSH, LH, PMSG, HCG), or on the pituitary (GnRH). However, none of the hormone treatments have been able to correct the catecholamine deficiency, which is the root cause of the problem.

In contrast to the hormone-based methods, the method of the present invention corrects the catecholamine deficiency in the hypothalamus, which in turn causes the estrous cycle to be reinitiated in both lab animals and in female dogs. Because the hormones, the neurotransmitters, and the various mechanisms that control the reproductive cycle and estrus (heat) are generally the same in all female mammals, the method of the present invention is applicable to all female mammals, including female humans.

In the present invention, the Deprenyl, its analogs, isomers, or salts, acts (1) as an inhibitor of monoamine oxidase, an enzyme involved in the degradation of neurotransmitters such as norepinephrine and dopamine, which results in an increase in the levels of these neurotransmitters, (2) directly on neurons in the hypothalamus to stimulate the release of these neurotransmitters, or (3) on the pituitary to stimulate the release of luteinizing hormone. Compounds related to deprenyl that can be used in the method of the present invention include selegiline (CAS 14611-51-9), selegiline hydrochloride (CAS 14611-52-0), and phenethylene, N, alpha-dimethyl-N-2-propynyl hydrochloride (CAS 2079-54-1). The present invention further includes other MAO inhibitors, which have an effect on reproduction, which can be used in combination with or instead of the deprenyl or its related compounds to reinstate the estrous cycle in female mammals, including human females, which have discontinued anti-fertility treatments or have impaired estrous for other reasons. Iproniazid (4-pyridinecarboxyylic acid, 2-(1-methylethyl)hydrazine), CAS 54-92-2, is an example of a MAO inhibitor that has been shown to reinstate the estrous cycle in acyclic old rats (Quadri et al., Neuroendocrinol. 11: 248-254 (1973)).

The method the present invention is particularly useful for reinstating the estrous cycle in a female mammal in which treatment with an anti-fertility composition has been discontinued. For example, female racing greyhounds that have been treated with testosterone for an extended period of time or women who have been on a contraceptive such as the pill, which comprises estrogen/progesterone, for an extended period of time, to inhibit the estrous cycle can be taken off the anti-fertility treatment and placed on a treatment that comprises a MAO such as deprenyl, an optimal isomer thereof, or a salt thereof. The MAO or deprenyl causes an increase in the neurotransmitters which stimulates or reinstates the estrous cycle in the female. The female with the reinstated estrous cycle is susceptible to fertilization and thus, pregnancy. Once the estrous cycle has been reinitiated or pregnancy has been established, the treatment with the MAO or deprenyl is preferably discontinued. The method is also useful for reinitiating the estrous cycle in female mammals where the anti-fertility treatment had bee discontinued for a period of time before the treatment with deprenyl.

The method of the present invention can also be used to enhance fertility in female mammals in which the female mammal has an impaired estrous cycle caused by decreased secretion of neurotransmitters such as norepinephrine, dopamine, or serotonin wherein the decrease in secretion has an organic cause or was caused by a disease. Thus, the present invention is useful for increasing the conception rate in female mammals such as canines, dairy cows, horses, or female humans in which the estrous cycle has been impaired by a decrease in the secretion of neurotransmitters by treatment with anti-fertility compositions or which has an organic basis or has been caused by disease.

The estrous cycle in a female mammal is reinstated by administering an effective amount of a composition comprising a MAO inhibitor such as deprenyl, an optimal isomer thereof, or a salt thereof, in a carrier to the female mammal. The composition can further comprise a mixture of MAO inhibitors or a mixture of deprenyl or related compound and one or more other MAOs in a carrier. Preferably, the carrier comprises inert or inactive ingredients. Further, the carrier that is used is a carrier which has been approved for use in the particular female mammal by the U.S. Food and Drug Administration or when the composition is intended for use in a foreign country, by the appropriate regulatory authority of the foreign country.

When the composition comprises deprenyl or a related compound, the composition is administered to the female mammal such that the deprenyl or related compound in the composition is administered to the female mammal in a dosage that is at least about 0.1 mg/kg of body weight of the female mammal. Alternatively, the deprenyl or related compound in the composition is at a dosage not more than about 1 mg/kg of body weight of the female mammal. When the composition comprises solely another MAO or a combination of deprenyl or related compound and one or more other MAOs or a mixture of other MAOs, the amount of the above MAO alone or combinations is that which is sufficient to reinstate the estrous cycle in the female animal. In particular embodiments, the above MAO alone or the combinations in the composition is at least about 0.1 mg/kg of body weight of the female animal or not more than about 1 mg/kg body weight of the female mammal.

Therefore, the present invention provides a method for reinstating the estrous cycle in a female mammal wherein the estrous cycle has been impaired for a period of time by treatment with a MAO such as deprenyl, an optimal isomer thereof, or a salt thereof, or a mixtures of MAO that cause an increase in the secretion of neurotransmitters from the brain. The impairment of the estrous cycle can be by administration of a composition such as testosterone or estrogen/progesterone or by compositions that affect the release of neurotransmitters such as norepinephrine, dopamine, or serotonin from the brain or the secretion of LH from the pituitary gland or the impairment can have an organic origin or have been caused by a disease. The administration can be discontinued just prior to the application of the method of the present invention or at some time period before the application of the method of the present invention.

The following examples are intended to promote a further understanding of the present invention.

### EXAMPLE 1

The greyhound racing industry encounters major problems with reinitiating estrous cycles in retired animals when they plan to use them for breeding. Because there are no practical methods for reinitiating estrous cycles in these animals, there has been considerable loss to the greyhound racing industry, in particular, the inability to breed greyhounds with desirable racing traits. In general, retired female greyhounds that are retired either do not come into heat at all or take such an enormous amount of time to come to heat that the dams encounter other problems such as dystocia, small litter size and loss of genetic potential. This example shows that using deprenyl according to the method of the present invention induces estrous in retired racing female greyhounds that have had their testosterone treatments discontinued prior to treatment with deprenyl.

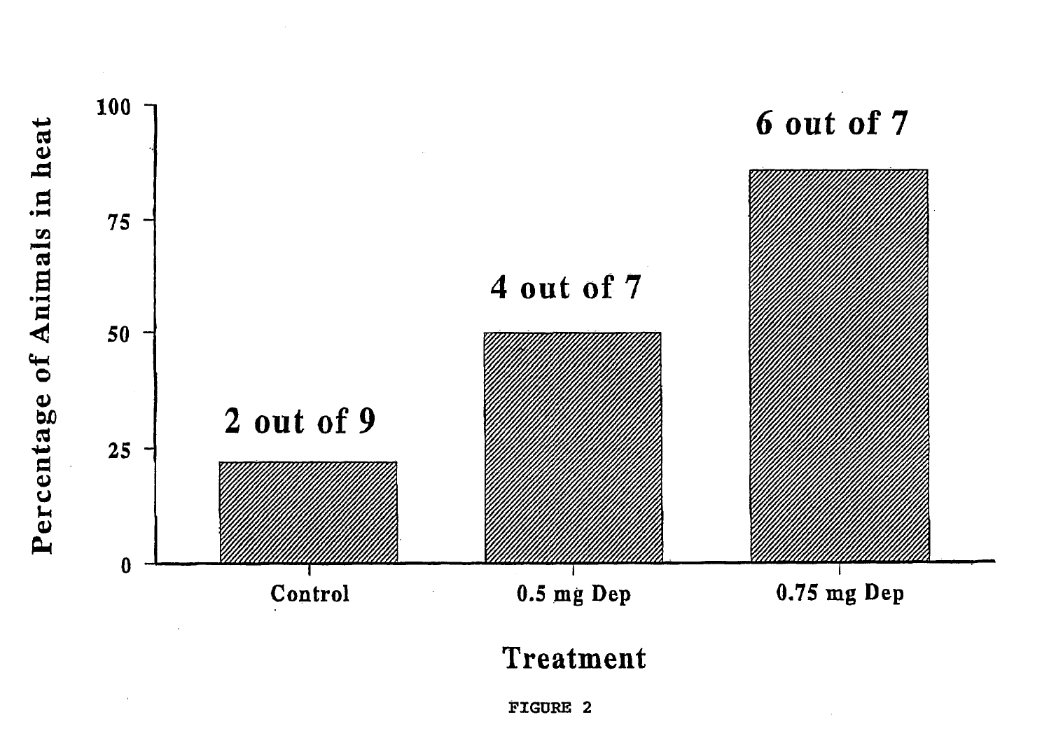

Retired racing female greyhounds that were previously treated with testosterone were used in the experiments. Nine animals were used in the control group, and seven animals each were used in the low dose (0.5 mg/kg body weight deprenyl) and the high dose (0.75 mg/kg body weight) groups. Control animals were treated with oral saline solution. Experimental, animals were treated with one of the two doses of deprenyl orally dissolved in saline. FIG. 2 shows the results.

As shown in FIG. 2, only 22% of the control animals came to heat after treatment with saline. In contrast to the control animals, a dose-dependent response was observed in animals that received the deprenyl treatment. Fifty percent of the animals came to heat after treatment with the low dose of Deprenyl and 86% of the animals came to heat with the higher dose of deprenyl. After coming into heat, the bitches are then impregnated with sperm to produce pregnancy.

The method of the present invention for reinitiating the estrus cycle in retired female greyhounds for breeding purposes, will have an important and positive effect on the greyhound racing industry not only in the United States but also in Australia, the United Kingdom, and other parts of Europe where greyhound racing is a popular sport.

### EXAMPLE 2

This example shows that deprenyl stimulates release of monoamines from the hypothalamus in vitro, which further explains the mechanisms involved in the present invention.

Deprenyl is an irreversible monoamine oxidase (MAO)-B inhibitor (Knoll et al. In: Advances in Biochemical Psycho-Pharmacology, Vol. 5. Costa and Sandler (eds.), Raven Press, New York, N.Y. 1972. pp. 393-407; Knoll, Acta Neurol. Scand. suppl. 95:57-80 (1983)). However, depending on the dose and duration of treatment it has also been shown to inhibit MAO-A at higher concentrations (Knoll et al. In Advances in Biochemical Psycho-Pharmacology, Vol. 5. Costa and Sandler (eds), Raven Press, New York, N.Y. 1972. pp. 393-407; Yang et al. J. Pharm. Exp. Therap. 180: 733-740 (1974)). Like some other MAO inhibitors, deprenyl was also used originally as an antidepressant (Knoll et al. In: Advances in Biochemical Psycho-Pharmacology, Vol. 5. Costa and Sandler (eds), Raven Press, New York, N.Y. 1972. pp. 393-407; Knoll, Acta Neurol. Scand. suppl. 95:57-80 (1983)). Recently, because of its ability to inhibit MAO and increase dopaminergic tone in the striatum, it is being used in the treatment of Parkinson's disease (Knoll, Mech. Aging Develop. 30: 109-122 (1985); Knoll, J. Neural Trans. (suppl) 25: 45-66 (1987); Tetrund et al., Science 245: 519-522 (1989)).

Deprenyl has also been shown to have other novel biological actions such as, increasing life-span and sexual activity in male rats (Knoll et al., Life Sci. 45: 525-531 (1989)). Recently, it was reported that deprenyl also inhibits carcinogen-induced mammary tumor growth in rats and reinitiates estrous-cycles and decreases the incidence of spontaneous mammary tumors and pituitary tumors in old acyclic rats (ThyagaRajan et al., Endocrinol. 136: 1103-1110 (1995)). Also, deprenyl decreases serum prolactin concentrations in young and old rats (MohanKumar et al., Life Sci. 54: 841-845 (1994);ThyagaRajan et al., Endocrinol. 136: 1103-1110 (1995)). However, the mechanism by which deprenyl produces these effects is not clear.

Brain neurotransmitters, especially monoamines are known to be involved in various central and neuroendocrine effects such as increases in sexual activity, life-span, reinitiation of estrous cycles and decreasing serum prolactin concentrations (Meites, Prog. Clin. Biol. Res. 75: 1-8 (1981); Meites, J. Reprod. Fertil. Suppl. 46:1-9 (1993); Meites, Acta Endocrinol. (Copenh.) 125 Suppl1: 98-103 (1991)). More specifically, monoamine changes in the hypothalamus appear to play a role in integrating these effects since the hypothalamus houses all the releasing hormone neurons that regulate these functions. Therefore, deprenyl by inhibiting MAOs produces its central and neuroendocrine effects by affecting monoamine release from the hypothalamus. Interestingly, very little is known about the effects of deprenyl on the release of hypothalamic monoamines. In this example, a combination of high performance liquid chromatography with electrochemical detection (HPLC-EC) and a static in vitro incubation system was used to investigate the direct effects of deprenyl on hypothalamus to affect monoamine release.

### MATERIALS AND METHODS

Animals

Four- to six-month old male Sprague-Dawley rats were obtained from Amitech (Omaha, Nebr.). These rats were housed in groups in air-conditioned (23±2° C.) and light controlled (lights on from 0700-1900 hours) animal rooms. They were provided rat chow and water ad libitum.

On the day of the experiment, the rats were sacrificed between 1200-1300 hrs. The brains were removed immediately and the hypothalami were dissected out using the following boundaries: the posterior part of the optic chiasmas the anterior limit, the anterior part of the mammillary bodies as the posterior limit, and the lateral hypothalamic sulci as the lateral limits (Palazzolo et al., Life Sci. 47: 2105-2109 (1990); Palazzolo et al., Life Sci. 51: 1797-1802 (1992)).

In vitro Incubation

The hypothalami were weighed and divided into two halves along the sagittal plane and placed in a 12×75 mm glass tube containing 300 μl of Krebs-Ringer Henseleit (KRH) solution. The hypothalami were incubated in a static in vitro incubation system as described previously (Palazzolo et al., Life Sci. 47: 2105-2109 (1990); Palazzolo et al., Life Sci. 51: 1797-1802 (1992)). Briefly, the incubation system consisted of a plexiglass water bath, a magnetic circulator fin, and a thermostat coil. The bath was filled with distilled water and the thermostat was switched on at least an hour before the perfusion. The temperature in the water bath was maintained at 37° C. The magnetically controlled circulator fins stirred the water constantly to maintain a uniform temperature throughout the water bath. The incubation medium, KRH, consisted of 117 mM NaCl, 4.7 mM KCl, 1.2 mM MgSO₄, 1.2 mM KH₂PO₄, 2.5 mM CaCl₂, 24.8 mM NaHCO₃, 11.1 mM glucose, 0.1 mg/ml ascorbic acid in 1 L of pyrogen free water. During the experiment, the incubation medium was supplied a mixture of 95% oxygen and 5% carbon dioxide constantly through gas inlet lines at a slow and steady rate to avoid turbulence. To stabilize the release of monoamines, each hypothalamus was preincubated in KRH for 60 min. Following this, the hypothalami were incubated for four 60-min periods as described below.

Treatment

During the first incubation period, the hypothalami were incubated in KRH for 60 min to measure the basal release of monoamines and their metabolites. During the second incubation period, the hypothalami were incubated in KRH containing 0, 0.1 mM, 1.0 mM, or 10 mM of deprenyl. After this, the hypothalami were rinsed and incubated in KRH alone (without deprenyl) during the third incubation period to measure the residual effects, if any, from the pervious incubation period. In the final incubation period, the hypothalami were incubated with high K⁺ KRH, which, had the same composition as the KRH except that it contained 60 mM KCl and 57 mM NaCl, to check the viability of the tissue. Hypothalami were rinsed with KRH in between incubations. After each incubation period, incubation medium was removed and 0.1 M HClO₄ was added to it at the ratio of 25:1 (v/v) and was stored at −70° C. until analysis for neurotransmitters by HPLC-EC. At the end of the treatment period, the hypothalami were transferred to microsample vials containing 0.1 M HClO₄ and were stored at −70° C. At the time of HPLC analysis, the hypothalami were homogenized in KRH and centrifuged at 1000 RPM for 15 min and the supernatant was used for the measurement of neurotransmitters.

High Performance Liquid Chromatography with Electrochemical Detection (HPLC-EC)

The HPLC-EC system has been described before (Palazzolo et al., Life Sci. 47: 2105-2109 (1990); Palazzolo et al., Life Sci. 51: 1797-1802 (1992); ThyagaRajan, S.; Meites et al., Endocrinol. 136: 1103-1110 (1995)). Briefly, it consisted of an LC-4B electrochemical detector (Bioanalytical Systems, West Lafayette, Ind.); a phase II, 5 μm ODS reverse phase, C-18 column; a glassy carbon electrode, a C-R6A Chromatopac integrator, a CTO-6A column oven, and an LC-6A pump (Shimadzu, Columbia, Md.). The mobile phase consisted of monochloroacetic acid (14.14 g/l), sodium hydroxide (4.675 g/l), octanesulfonic acid disodium salt (0.3 g/l), ethylenediaminetetraacetic acid (0.25 g/l), acetonitrile (3.5%), and tetrahydrofuran (1.4%). The mobile phase was made in pyrogen-free water and then filtered and degassed through the Milli-Q purification system (Millipore Co., Bedford, Mass.). It was pumped through the HPLC system at a flow rate of 1.7 ml/min. The sensitivity of the detector was 1 nA full scale, and the potential of the working electrode was 0.65 V. The column and the electrodes were kept in a column oven maintained at 37° C. At the time of HPLC analysis, the incubation medium was diluted at the ratio of 1:4 with 0.1 M HClO₄. Fifty μl of the diluted incubation medium along with 25 μl of the internal standard (0.05 M isoproterenol) was injected into the HPLC system.

Statistical Analysis

Differences in the release of neurotransmitters during various incubation periods in each group was analyzed by ANOVA followed by Fischer's LSD. Differences in the contents of neurotransmitters in the hypothalamus among the various groups were analyzed by one way ANOVA followed by Fischer's LSD.

### RESULTS

Norepinephrine

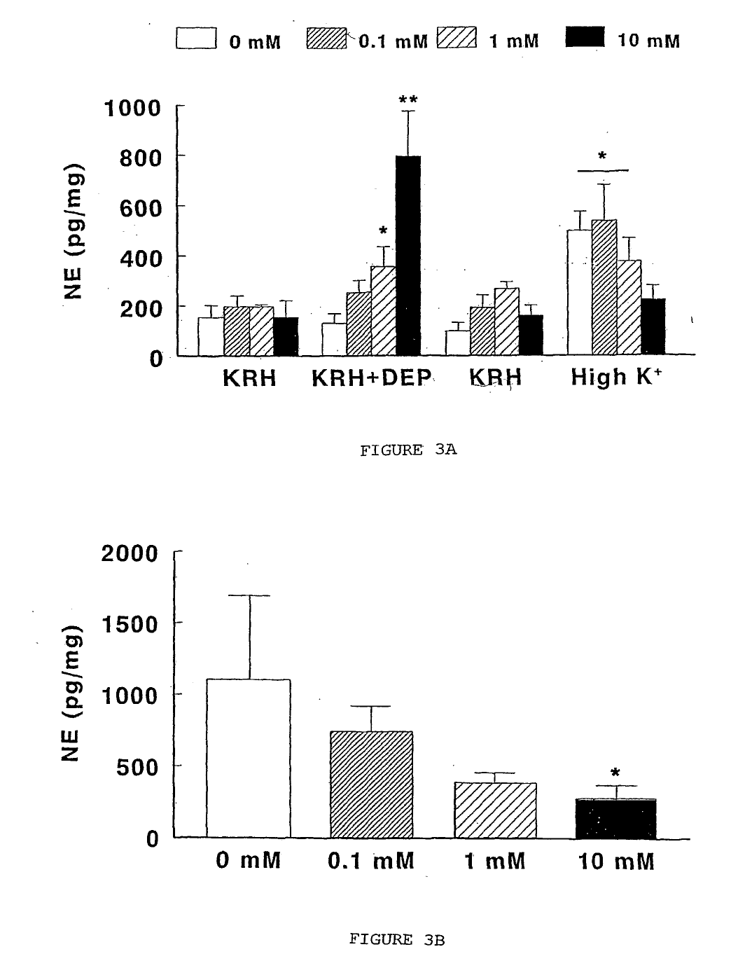

The effects of deprenyl on the release of norepinephrine (NE) are shown in FIG. 3A. The pretreatment release of NE in the three treatment groups were not different from each other. In the control group (0 mM), NE release (Mean±S.E.; pg/mg hypothalamus;153.4±49) did not change significantly during the first 3 incubation periods but produced a marked increase of 497.9±76 when incubated with high potassium KRH (p<0.05). In contrast to the control group, incubation with different doses of deprenyl produced a dose-dependent increase in NE release. When the hypothalami were incubated with 0.1 mM deprenyl, NE release seemed to increase from pretreatment levels of 196.9±43 to 252.3±48, but this increase was not significant. NE release returned to pretreatment levels (185.1±42) during the third incubation period. Once again challenge with high potassium KRH produced a robust increase in NE release (538.2±142; p<0.05).

Incubation with 1 mM deprenyl on the other hand, increased NE release significantly from 166.4±25 to 354.7±:79 during the second incubation period (p<0.05) and decreased to 267.7±28 during the third incubation period. High K+ KRH once again stimulated NE release (375.2±92) during the last incubation period (p<0.05). Incubation with 10 mM deprenyl produced a robust increase in NE release during the second incubation period (799.9±141) when compared to the pretreatment incubation (132.8±56; p<0.05) which declined to 159.8±41 during the third incubation period. However, incubation with high K⁺ KRH produced only a modest increase (222.4±57) which was not statistically significant.

The content of NE in the hypothalami at the end of the experiment is shown in FIG. 3B. Hypothalami in the control and 0.1 mM deprenyl treated groups had significantly higher concentrations of NE (1104±588 and 743.3±179 respectively). These were nearly 3-4 fold higher than the NE concentrations in the 1 mM and 10 mM deprenyl treated groups (388.1±71 and 281.3±91 respectively; p<0.05).

Dopamine

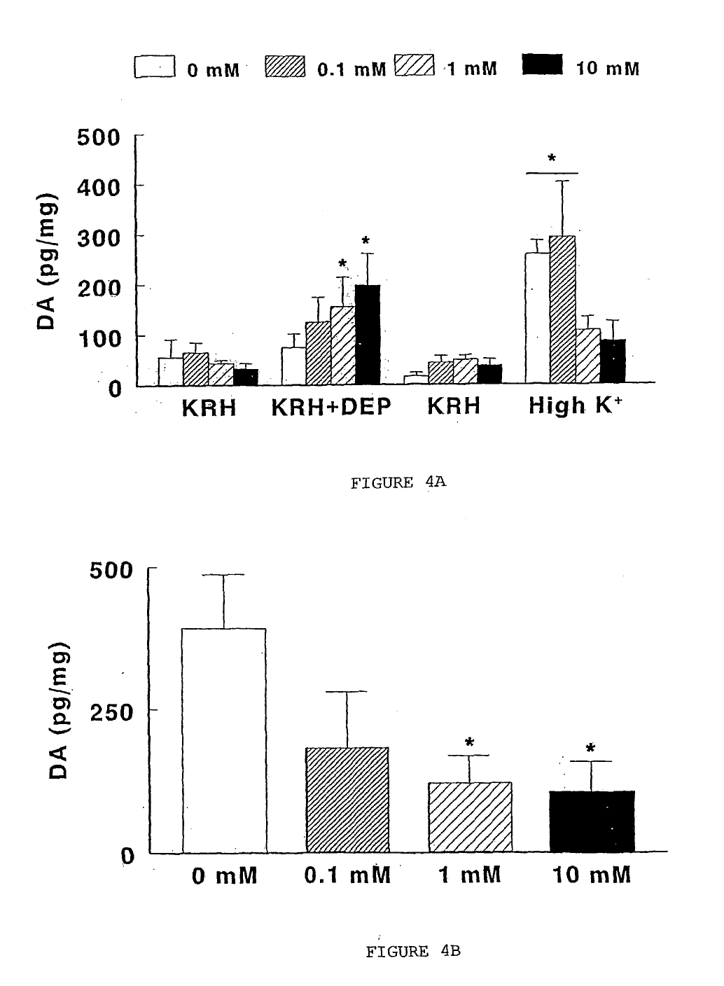

The effects of deprenyl on dopamine (DA) release are shown in FIG. 4A. In contrast to NE release, DA release seemed to decrease much rapidly over time. It decreased from 74.6±27 in the second incubation period in the control group to 16.1±17 in the third incubation period (p<0.01). Incubation with high K⁺ KRH in this group produced a significant increase in DA release of 158.9±27 (p<0.01). Similar to its effects on NE, deprenyl produced a dose-dependent increase in DA release. Incubation with 0.1 mM deprenyl seemed to increase DA release (125.1±49) but this was not significantly different from the pretreatment release rate. Treatment with 1 mM deprenyl on the other hand produced a sharp increase in DA release (154.8±59) when compared to pretreatment levels (42.9±6). In both these groups, removal of deprenyl from the medium during the third incubation period brought DA release to pretreatment levels and stimulation with high K⁺ KRH produced significant increases (393.1±109 and 106.8±27 respectively) in DA release. Although incubation with 10 mM deprenyl produced a stark increase in DA release (197.7±63) and removal of deprenyl from the medium returned DA release to basal levels as in the other groups, stimulation with high potassium KRH produced an increase (85.3±40) that was not statistically significant from the pretreatment levels.

DA content of the hypothalami in all the groups at the end of the experiment is shown in FIG. 4B. DA concentrations in the 1 mM and 10 mM groups were significantly less compared to the 0 mM group (p<0.05).

DOPAC

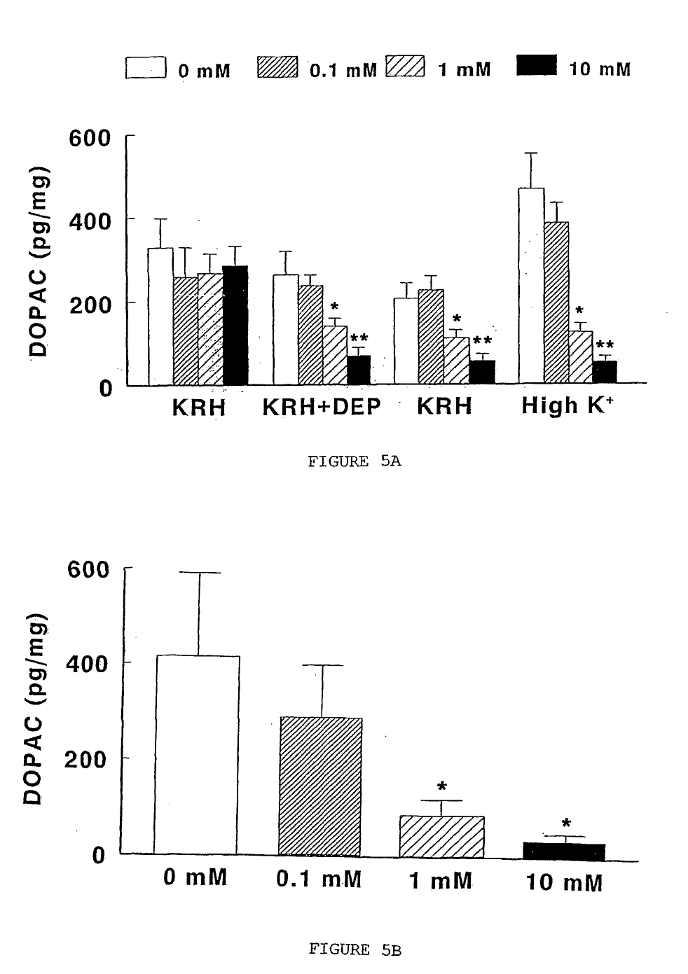

The dose-dependent inhibitory effects of deprenyl on DOPAC release are shown in FIG. 5A. DOPAC release decreased during the third incubation (205.3±38) when compared to the pretreatment level (329.3±70) in the control group but increased upon stimulation with high potassium KRH (467.3±84; p<0.05). Incubation with 0.1 mM deprenyl did not produce any change in DOPAC release but incubation with 1 mM deprenyl produced a marked decrease in DOPAC release (139.6±19) when compared to pretreatment levels (267.7±46; p<0.01). A more pronounced effect was observed after incubation with 10 mM deprenyl. DOPAC release decreased from pretreatment levels of 285.6±28 to 68.9±20 (p<0.001). In both these groups, DOPAC release remained low even after removal of deprenyl from the medium. Moreover, stimulation with high potassium KRH did not produce any increase in DOPAC release in all the deprenyl-treated groups. The concentration of DOPAC in the hypothalamus at the end of the experiment (FIG. 5B) was markedly reduced in the 1 and 10 mM deprenyl treated groups {86.8±34 and 34.3±16 respectively) when compared to the control and the 0.1 mM deprenyl treated groups (416±176 and 290.0±110 respectively; p<0.05).

5-HT

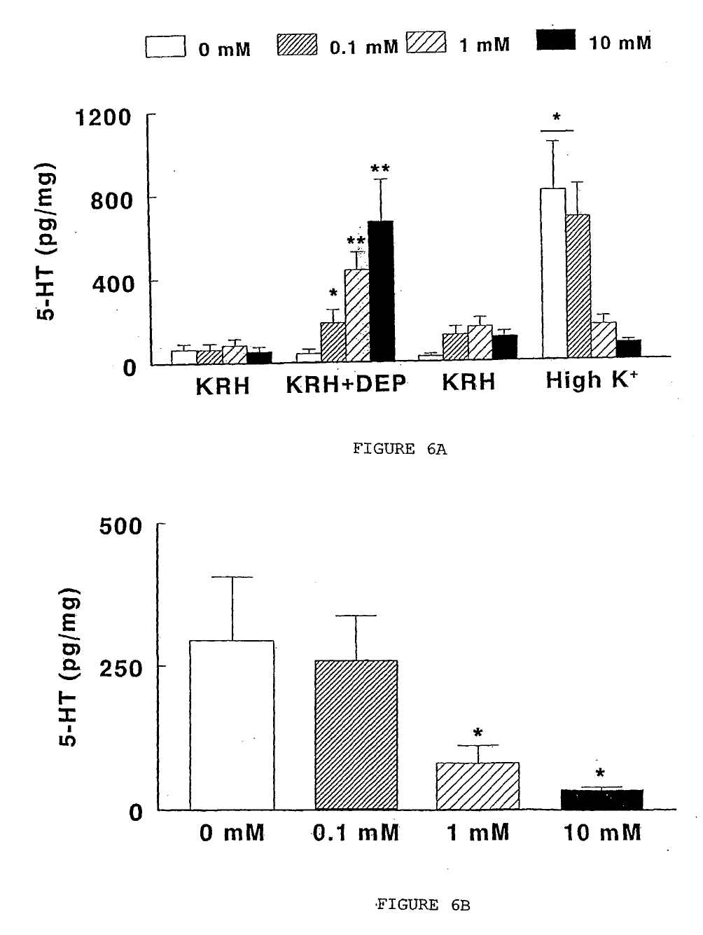

The basal release of 5-HT as observed in the control group decreased significantly with time (FIG. 6A). It decreased from 65.1±20 in the first incubation period to 41.0±21 and further declined to 23.2±12 during the third incubation period (p<0.05). Upon incubation with different doses of deprenyl, a remarkable and dose-dependant increase in 5-HT release was observed {186.4±62, 438.4±85, and 670.3±199 with 0.1, 1, and 10 mM deprenyl, respectively). The release of 5-HT declined rapidly upon removal of deprenyl from the medium in the third incubation period. Stimulation with high potassium KRH produced a significant increase in 5-HT release in 0 mM and 0.1 mM groups. Similar to NE and DA, content of 5-HT at the end of the experiment was significantly reduced in 1 mM and 10 mM groups compared to the control group (FIG. 6B; p<0.05).

5-HIAA

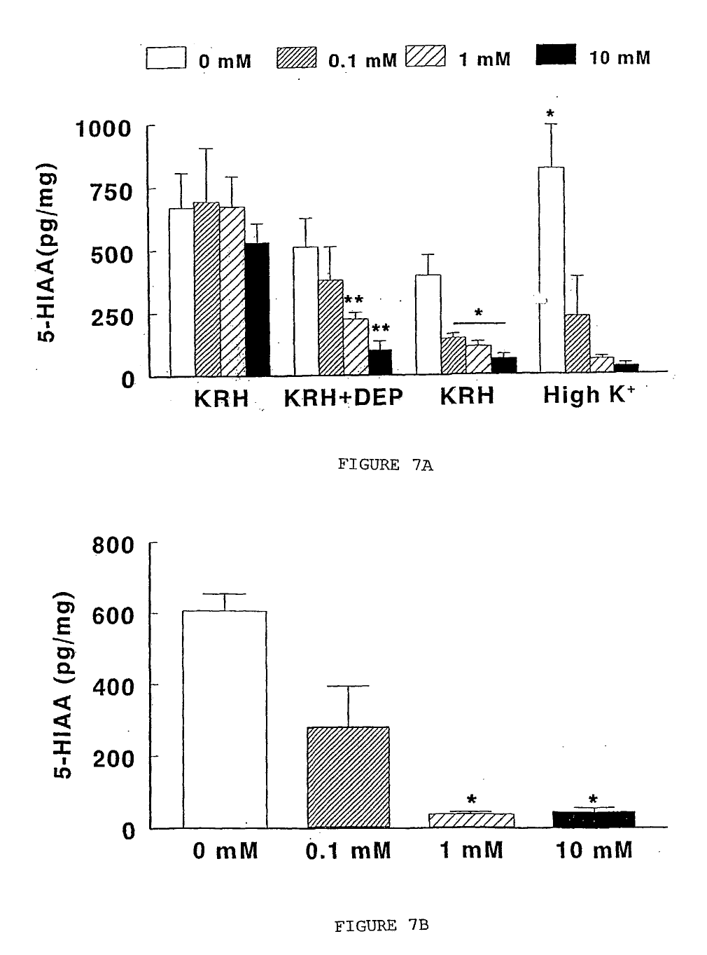

In contrast to the release of 5-HT, the release of its metabolite, 5-HIAA, showed a dose-dependant decrease when the hypothalami were incubated with different doses of deprenyl or KRH (FIG. 7A). The inhibition of 5-HIAA release observed over time continued even after stimulation with high potassium KRH. Moreover the content of 5-HIAA in the hypothalami at the end of the experiment (FIG. 7B) was significantly low in the deprenyl treated groups when compared to the control group (P<0.05).

### DISCUSSION

The results clearly demonstrate for the first time that deprenyl stimulates the release of NE, DA and 5-HT from the hypothalamus in vitro in a dose-dependant fashion. The release of these neurotransmitters either remains stable or decreases gradually as a function of time. This is a characteristic feature of the in vitro incubation system and has been observed in other studies (Palazzolo et al., Life Sci. 47: 2105-2109 (1990); Palazzolo et al., Life Sci. 51: 1797-1802 (1992)). The consistent finding in all the groups was that deprenyl not only prevented this decrease but produced a marked increase in the release of NE, DA and 5-HT. Stimulation with high potassium KRH produced a pronounced increase in the release of these neurotransmitters indicating that the tissues were viable. In contrast, the release of the metabolites, DOPAC and 5-HIAA are inhibited upon incubation with deprenyl clearly indicating its ability to inhibit MAO. These results are supported by another study in which deprenyl increased NE content in the teldiencephalon and the rest of the brain (Zsilla et al., In: Typical and Atypical Antidepressants: Molecular Mechanisms. Costa and Racagni (eds.). Raven Press, New York, N.Y. 1982). Recent studies from our laboratory have shown that deprenyl can increase the concentration of NE in the striatum and the mediobasal hypothalamus and stimulate the release of NE from the mediobasal hypothalamus in vivo (ThyagaRajan et al., Endocrinol. 136: 1103-1110 (1995); ThyagaRajan et al., Neurosci. Lett. 270: 79-82 (1999)). The present study demonstrates that deprenyl stimulates NE release through its direct action on the hypothalamus. There are several indirect lines of evidence to indicate that this may be the case and provide explanations for some of the novel effects of deprenyl.

Deprenyl is known to increase sexual activity in male rats and reinitiate estrous cycles in old female rats. Both of these effects are known to involve luteinizing hormone (LH), the release of which from the pituitary is also known to be stimulated by deprenyl (MohanKumar et al., Life Sci. 61 : 1783-1788 (1997)). NE is one of the important neurotransmitters involved in the regulation of LH secretion. The fact that the synthesis, release and content of NE declines in the hypothalamus of old animals is well established (Meites, J. Reprod. Fertil. Suppl. 46:1-9 (1993); Meites, Acta Endocrinol.(Copenh) 125 Suppl1: 98-103 (1991)). I-dopa and the MAO inhibitor iproniazid, both of which are compounds known to increase brain NE levels, could reinitiate estrous cycles in old rats (Quadri et al., Neuroendocrinol. 11: 248-254 (1973)). Recently, we have demonstrated that administration of deprenyl itself could reinitiate estrous cycles in old female rats (ThyagaRajan et al., Endocrinol. 136: 1103-1110 (1995)) indicating that this effect of deprenyl is most probably mediated through an increase in NE release. The current study provides the first direct evidence for this phenomenon.

Incubation of the hypothalamus with deprenyl also increased DA release in a dose-dependant manner. This finding is supported by another study in which direct infusion of deprenyl into the hypothalamus stimulated the release of DA (ThyagaRajan et al., Neurosci. Lett. 270:79-82 (1999)). Other studies have also indicated that deprenyl increases the turnover rate and release of DA from the striatum (Harsing Jr. et al., Br. J. Pharmacol. 83: 741-749 (1984); Knoll, J. Neural Trans. (suppl) 25: 45-66 (1987). Other indirect lines of evidence suggest that deprenyl may indeed produce this effect in the hypothalamus. Deprenyl has been shown to inhibit prolactin secretion (MohanKumar et al., Life Sci. 61: 1783-1788 (1997); ThyagaRajan et al., Endocrinol. 136: 1103-1110 (1995)) which could be mediated through stimulation of DA since prolactin is under the inhibitory control of DA (Meites, Prog. Clin. Biol. Res. 75: 1-8 (1981)). Moreover, deprenyl has been shown to cause the regression of prolactin-dependant mammary tumors (ThyagaRajan et al., Endocrinol. 136: 1103-1110 (1995); ThyagaRajan et al., Cancer Lett. 123: 177-183 (1998): ThyagaRajan et al., Endocrine 10: 225-232 (1999)). This effect is probably achieved through an increase in DA levels which causes suppression of prolactin secretion which ultimately results in the regression of the mammary tumors. Therefore it is conceivable that deprenyl may indeed stimulate the release of DA to produce some of its beneficial effects. After incubation with the highest dose of deprenyl, the release of NE, DA and 5-HT were unaltered upon stimulation with high K⁺ KRH. This may be due to the exhaustion of the existing pool of neurotransmitters in the hypothalamic blocks. This conclusion is supported by the observation that the content of these neurotransmitters at the end of the experiment were also significantly low in the 10 mM group when compared to the control group.

Apart from stimulating the release of NE and DA from the hypothalamus, the present study provides evidence that deprenyl stimulates the release of 5-HT in a dose-dependent fashion. Like NE and DA, 5-HT is also involved in a number of neuroendocrine effects including the regulation of LH and prolactin (Levy et al., Front. Neuroendocrinol. 15: 85-156 (1994); Yatham et al., Life Sci. 53: 447-463 (1993)). Thus, it is quite probable that at least some of the effects of deprenyl could be mediated through its effects on 5-HT in the hypothalamus.

In contrast to its effect on NE, DA and 5-HT, deprenyl decreased the release of the metabolites DOPAC and 5-HIAA in a dose-dependant fashion. In rats, the metabolism of monoamines is a complex process and is achieved by both forms of monoamine oxidases (MAO-A and MAO-B) (Yang et al., J. Pharm. Exp. Therap. 180:7 33-740 (1974); Youdim et al., J. Neural Transm. Gen. Sect. 91: 181-195 (1993)). While DA acts as a substrate for both MAO-A and B, 5-HT is metabolized by MAO-A (Salach et al., Mol. Pharm. 16: 234-241 (1979); Schoepp et al., Biochem. Pharm. 31 :2961-2968 (1982)). Thus, deprenyl being a MAO-B inhibitor inhibited the metabolism of DA and decreased the levels of its metabolite significantly. The interesting finding in the present study was the capacity of deprenyl to also inhibit the levels of 5-HIAA, even at the lowest dose used. As alluded to before, deprenyl could act on both forms of MAO (Ekstedt et al., Biochem. Pharmacol. 28: 919-923 (1979); Waldmeier et al., Biochem. Pharmacol. 27: 801-802 (1979)), hence, deprenyl could simultaneously inhibit the release of both DOPAC and 5-HIAA. A similar decrease in DOPAC and 5-HIAA concentrations in the mediobasal hypothalamus and the striatum have been observed before after deprenyl treatment (ThyagaRajan et al., Endocrinol. 136: 1103-1110 (1995); ThyagaRajan et al., Endocrine 10: 225-232 (1999)). The inhibition observed in the present study was so great that high K⁺ KRH, which was capable of stimulating the release of NE, DA and 5-HT, was still unable to stimulate the release of DOPAC and 5-HIAA. This could only mean that deprenyl totally inhibited the break-down of DA and 5-HT to its metabolites while simultaneously stimulating the release of these neurotransmitters from the hypothalamus. This is not surprising since deprenyl is an established monoamine oxidase inhibitor which acts on the flavin binding site to irreversibly inhibit this enzyme (Maycock et al. In: Monoamine Oxidase and Its Inhibitors. Wolstenholme and Knight (eds.) Elsevier, North Holland. 1976. pp. 33-47). The enzyme activity is believed to be reinstated only after the synthesis of new enzyme molecules (Maycock et al. In: Monoamine Oxidase and Its Inhibitors. Wolstenholme and Knight (eds.) Elsevier, North Holland. 1976. pp. 33-47) which is not possible in the in vitro model. This clearly explains the stark decrease in the release of DOPAC and 5-HIAA after deprenyl treatment.

The mechanism by which deprenyl stimulates the release of NE, DA, 5-HT is not known. It has been suggested that it is an indirectly acting amine that affects the peripheral nervous system (Simpson, Biochem. Pharm. 27: 1591-1595 (1978)). It may enter the neuronal membrane through passive diffusion or act as a substrate for membrane pumps for neurotransmitters such as NE. Upon entering the neuron, it then acts to evoke the release of neurotransmitters (Simpson, Biochem. Pharm. 27: 1591-1595 (1978)). The data in support deals exclusively with the peripheral nervous system but suggests that deprenyl could act in a similar fashion in the central nervous system. Regardless of the mechanism, the capacity of deprenyl as a stimulator of neurotransmitter release in itself as demonstrated by this study is potentially a new feature which could explain the novel actions of this drug.

While the present invention is described herein with reference to illustrated embodiments, it should be understood that the present invention is not limited hereto. Those having ordinary skill in the art and access to the teachings herein will recognize additional modifications and embodiments within the scope thereof. Therefore, the present invention is limited only by the claims attached herein.

We claim:

1- A method for inducing pregnancy in a female mammal wherein the estrous cycle of the female mammal has been inhibited by regular treatment for a period of time with an estrous cycle-inhibiting composition to inhibit the pregnancy, which has been discontinued leaving the female mammal unable to become pregnant, the steps comprising:
(a) orally administering an effective amount of a composition comprising deprenyl, an optimal isomer thereof, or a salt thereof to the female mammal to reinitiate the estrous cycle in the female mammal; and
(b) impregnating the female mammal with sperm to induce the pregnancy.

2- The method of claim 1 wherein the mammal is a human.

3- The method of claim 1 wherein the mammal is a non-human (lower) mammal.

4- A method for bringing a canine bitch into heat for breeding wherein the estrous cycle of the bitch canine had been inhibited by the regular treatment for a period of time with an estrous cycle-inhibiting composition which has been discontinued leaving the bitch canine unable to breed, which comprises:
(a) orally administering a heat-inducing amount of a composition comprising deprenyl, an optimal isomer thereof, or a salt thereof to the canine bitch to bring the canine bitch into heat; and
(b) breeding the canine bitch.

5- The method of claim 4 wherein the canine bitch is a female racing greyhound.

6- The method of claim 5 wherein the racing greyhound has been retired from racing.

7- The method of any one of claims 4 to 6 wherein the deprenyl is administered at a dosage no more than about 1 mg/kg of body weight.

8- The method of any one of claims 4 to 6 wherein the deprenyl is administered at a dosage of at least about 0.1 mg/kg of body weight.

9- In a method for regulating the estrus cycle in a female mammal which had been regularly administered an estrus cycle-inhibiting composition to inhibit the estrus cycle in the female mammal for a period of time and which has been discontinued leaving the female mammal unable to become pregnant, the improvement comprising:
orally administering an effective amount of a composition comprising deprenyl, an optimal isomer thereof, or a salt thereof to the female mammal to reinitiate the estrous cycle in the female mammal.

10- The method of claim 9 wherein the mammal is a human.

11- The method of claim 9 wherein the mammal is a mammal other than a human.

12- The method of claim 11 wherein the mammal is a canine.

13- The method of claim 12 wherein the canine is a racing greyhound.

14- The method of claim 13 wherein the racing greyhound has been retired from racing.

15- The method of any one of claims 9 to 14 wherein the deprenyl is administered at a dosage no more than about 1 mg/kg of body weight.

16- The method of any one of claims 9 to 14 wherein the deprenyl is administered at a dosage of at least about 0.1 mg/kg of body weight.

17- In a method for reinitiating a reproductive cycle in a female mammal wherein the reproductive cycle has been inhibited by treatment with an estrous cycle-inhibiting composition for a period of time and which has been discontinued leaving the female mammal unable to become pregnant, the improvement which comprises:
orally administering an effective amount of a composition comprising deprenyl, an optimal isomer thereof, or a salt thereof to the female mammal to reinitiate the reproductive cycle in the female mammal.

18- The method of claim 17 wherein the mammal is a human.

19- The method of claim 17 wherein the mammal is a mammal other than a human.

20- The method of claim 17 wherein the mammal is a canine.

21- The method of claim 20 wherein the canine is a racing greyhound.

22- The method of claim 21 wherein the racing greyhound has been retired from racing.

23- The method of any one of claims 15 to 20 wherein the deprenyl is administered at a dosage no more than about 1 mg/kg of body weight.

24- The method of any one of claims 15 to 20 wherein the deprenyl is administered at a dosage of at least about 0.1 mg/kg of body weight.

25- In a method for reinitiating a reproductive cycle in a female mammal wherein the reproductive cycle has been inhibited by treatment with an estrous cycle-inhibiting composition for a period of time and which has been discontinued leaving the female mammal unable to become pregnant, the improvement which comprises:
orally administering an effective amount of a composition comprising one or more monoamine oxidase (MAO) inhibitors to reinitiate the reproductive cycle in the female mammal.

26- The method of claim 25 wherein the mammal is a human.

27- The method of claim 25 wherein the mammal is a mammal other than a human.

28- The method of claim 25 wherein the mammal is a canine.

29- The method of claim 28 wherein the canine is a racing greyhound.

30- The method of claim 29 wherein the racing greyhound has been retired from racing.

31- The method of any one of claims 25 to 30 wherein the monoamine oxidase (MAO) inhibitor is selected from deprenyl, selegiline, selegiline hydrochloride, and phenethylene, N, alpha-dimethyl-N-2-propynyl hydrochloride, and iproniazid.

32- The method of claim 31 wherein the deprenyl is administered at a dosage no more than about 1 mg/kg of body weight.

33- The method of claim 31 wherein the deprenyl is administered at a dosage of at least about 0.1 mg/kg of body weight.
